(19) United States
(12) Patent Application Publication
(10) Pub. No.: US 2022/0389022 A1
(43) Pub. Date: Dec. 8, 2022

(54) SOLID FORMS OF PONATINIB HYDROCHLORIDE AND PROCESS THEREOF

(71) Applicant: DR. REDDY'S LABORATORIES LIMITED, Hyderabad (IN)

(72) Inventors: Deepika PATHIVADA, Hyderabad (IN); Sharmistha PAL DATTA, Bangalore (IN)

(21) Appl. No.: 17/835,480

(22) Filed: Jun. 8, 2022

(30) Foreign Application Priority Data
Jun. 8, 2021 (IN) 202141025509
Aug. 29, 2021 (IN) 202141039105

(51) Int. Cl. C07D 487/04

(52) U.S. Cl. CPC C07D 487/04; C07B 2200/13

(57) ABSTRACT

The present application relates to crystalline Form P1 and Form P3 of Ponatinib hydrochloride and process for its preparation. The said forms are also used for making pharmaceutical composition of Ponatinib hydrochloride.

This application claims priority from Indian provisional application 202141025509, filed Jun. 8, 2021 and Indian provisional application 202141039105, filed Aug. 29, 2021, all of which are herein incorporated by reference in their entireties.

### FIELD OF THE INVENTION

Aspects of the present application relate to solid state form of Ponatinib hydrochloride. Specific aspects relate to crystalline Form P1, Form P3 of Ponatinib hydrochloride and process for its preparation.

### BACKGROUND OF THE INVENTION

The compound ponatinib (“compound 1”) is also known under the tradename Iclusig and was previously designated as AP24534. It is an FDA approved oral drug candidate developed by ARIAD Pharmaceuticals for the treatment of chronic myeloid leukemia (CML) and Philadelphia chromosome positive (Ph+) acute lymphoblastic leukemia (ALL). Generally speaking, the multi-kinase inhibitor is known to be useful in the treatment of disorders associated with pathological cellular proliferation, such as neoplasms, cancer, and conditions associated with pathological angiogenesis. The compound is a multi-target tyrosine-kinase inhibitor [W S Huang et al. (2010); Journal of Medical Chemistry 53 (12): 4701-19].

The compound was also designated as “3-(2-Imidazo[1,2-b]pyridazin-3-ylethynyl)-4-methyl-N-[4-[(4-methylpiperazine-1-yl)methyl]-3-(trifluoromethyl)phenyl] benzamide” or as “3-(imidazo[1,2-b]pyridazin-3-ylethynyl)-4-methyl-N-(4-((4-methylpiperazine-1-yl)-methyl)-3-(trifluoromethyl)phenyl)benzamide”, depicted below:

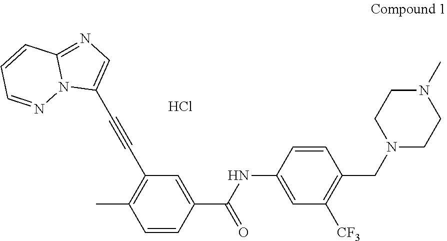 [SMILES: Cc1ccc(C(=O)Nc2ccc(CN3CCN(C)CC3)c(C(F)(F)F)c2)cc1C#Cc1cnc2cccnn12 ; Cl]

WO 2007/075869 A2 describes the synthesis of ponatinib (Example 16). Equivalent U.S. Pat. No. 8,114,874 is cited in the FDA Orange Book for ponatinib. WO2011/053938 A1 claims an oral formulation comprising ponatinib which can be used for the treatment of neoplasms, cancers and hyperproliferative disorders e.g. chronic myelogenous leukemia (CML). The document also describes the synthesis of ponatinib and its mono hydrochloride salt (pages 42-44 of the document). According to the disclosure, the hydrochloride salt of ponatinib was found to be an anhydrous crystalline solid.

International Patent Application Publication Nos WO2014/093579A2, WO2015/001098A1, WO2018/232501A1, and U.S. Pat. No. 10,221,184, reported various polymorphic forms of Ponatinib Hydrochloride.

Polymorphism is very common among pharmaceutical substances. It is commonly defined as the ability of any substance to exist in two or more crystalline phases that have a different arrangement and/or conformation of the molecules in the crystal lattice. Polymorphism, the occurrence of different crystal forms, is a property of some molecules and molecular complexes. A single molecule, like Ponatinib or salts thereof, may give rise to a variety of polymorphs having distinct crystal structures and physical properties like melting point, thermal behaviors (e.g. measured by thermogravimetric analysis—“TGA”, or differential scanning calorimetry—“DSC”), powder X-ray diffraction (PXRD) pattern, infrared absorption and solid state NMR spectrum. One or more of these techniques may be used to characterize a particular polymorph and to distinguish different polymorphic forms of a compound.

Different solid state forms (including solvated forms) of an active pharmaceutical ingredient may possess different properties. Such variations in the properties of different solid state forms and solvates may provide a basis for improving certain aspects of the API, such as its formulation, for example, by facilitating better processing or handling characteristics, changing the dissolution profile in a favorable direction, or improving stability (polymorph as well as chemical stability) and shelf-life. These variations in the properties of different solid state forms may also offer improvements to the final dosage form, for instance, if they serve to improve bioavailability. Different solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Pharmaceutical stability is believed to depend on simultaneous influence of various factors, of which some important factors are the sizes of the crystals, shape of the crystals, water content, residual solvents and impurities. Towards this end, it has been the endeavor of pharmaceutical scientists to provide novel salts and/or novel stable polymorphs of salts of drug substances, which would have the strengths of the crystalline forms, viz. thermodynamic stability, and those of the amorphous form, viz. enhanced solubility, rapid onset of action and an enhanced bioavailability.

Discovering new polymorphic forms and solvates of a pharmaceutical product can provide materials having, inter alia, desirable processing properties, such as ease of handling, ease of processing, chemical and polymorphic stability upon storage and processing, and ease of purification or are useful as advantageous intermediate crystal forms that facilitate conversion to other solid state forms (including other solvates) of said pharmaceutical product.

New polymorphic forms and solvates of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. Lastly, new polymorphic forms may be prepared with improved reliability and reproducibility compared to other forms, for example, in terms of crystallinity or polymorphic purity.

The intestinal tract obeys different pH ranges, which vary from pH 1 to 2 of the empty stomach, pH 3-5 for stomach filled with chyme, to pH 5-7.5 of the colon. A dissolution screening test regime for solid state forms of an active ingredient may be set up for example at pH 1.2, pH 4.5 and pH 6.8 in order to cover a broad range of the gastro-intestinal tract.

It is therefore an object of the present invention to provide solid state forms of ponatinib hydrochloride, as well as pharmaceutical compositions comprising the same, which do not show at least one or more of the above-described problematic properties. In particular, it is an object to provide solid state forms of ponatinib hydrochloride which show good bioavailability, low inter-patient variability, excellent overall therapeutic efficacy, excellent polymorphic and/or chemical stability, excellent flow properties, good compressibility, an excellent dissolution profile, and which are non-hygroscopic and/or do not electrostatically charge. The present inventors have found a solid state form of ponatinib hydrochloride which shows advantageous properties in at least one of the mentioned aspects.

### SUMMARY OF THE INVENTION

The present invention provides a crystalline Ponatinib Hydrochloride Form P1, processes for the preparation thereof, and pharmaceutical compositions and formulations comprising the solid state form of Ponatinib Hydrochloride Form P1, and processes for the preparation of the pharmaceutical compositions and formulations.

The present invention also provides the use of said Ponatinib Hydrochloride Form P1 for the manufacture of pharmaceutical compositions and formulations. Accordingly, the present invention further provides a pharmaceutical composition comprising said solid state Form P1 of Ponatinib Hydrochloride of the present invention. The pharmaceutical composition may additionally comprise at least one pharmaceutically acceptable excipient to form a pharmaceutical formulation that can, for example, be administered to patients in need of such treatment.

The present invention comprises a process for preparing the above-mentioned pharmaceutical formulations. The process comprises combining the solid state Form P1 of Ponatinib Hydrochloride with at least one pharmaceutically acceptable excipient.

The present invention provides a crystalline Ponatinib Hydrochloride Form P3, processes for the preparation thereof, and pharmaceutical compositions and formulations comprising the solid state form of Ponatinib Hydrochloride Form P3, and processes for the preparation of the pharmaceutical compositions and formulations.

The present invention also provides the use of said Ponatinib Hydrochloride Form P3 for the manufacture of pharmaceutical compositions and formulations. Accordingly, the present invention further provides a pharmaceutical composition comprising said solid state Form P3 of Ponatinib Hydrochloride of the present invention. The pharmaceutical composition may additionally comprise at least one pharmaceutically acceptable excipient to form a pharmaceutical formulation that can, for example, be administered to patients in need of such treatment.

The present invention comprises a process for preparing the above-mentioned pharmaceutical formulations. The process comprises combining the solid state Form P3 of Ponatinib Hydrochloride with at least one pharmaceutically acceptable excipient.

The solid state form as defined herein as well as the pharmaceutical compositions and formulations of Ponatinib Hydrochloride can be used as medicaments, particularly for the treatment of adults with chronic phase, accelerated phase, or blast phase chronic myeloid leukemia (CML) that is resistant or intolerant to prior tyrosine kinase inhibitor therapy, or Philadelphia chromosome positive acute lymphoblastic leukemia (Ph+ALL) that is resistant or intolerant to prior tyrosine kinase inhibitor therapy comprising administering a therapeutically effective amount of the crystalline Form P1 of Ponatinib Hydrochloride of the present invention, or a therapeutically effective amount of at least one of the pharmaceutical compositions or formulations of the present invention comprising said Ponatinib Hydrochloride Form P1 of the present invention to a patient in need thereof.

The present invention also provides the use of said s Ponatinib Hydrochloride Form P1, or at least one of the above pharmaceutical compositions and/or formulations for the manufacture of a medicament for treatment of chronic myeloid leukemia (CIVIL) and Philadelphia chromosome positive (Ph+) acute lymphoblastic leukemia (ALL).

Further, depending on which other solid state form it is compared with, the solid form of Ponatinib Hydrochloride Form P1 and Form P3 of the present invention may have advantageous properties selected from at least one of: chemical or polymorphic purity, increased crystallinity, flowability, solubility, dissolution rate, bioavailability, morphology or crystal habit, specific surface and bulk/tap density, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, and bulk density.

### BRIEF DESCRIPTION OF THE DRAWINGS

FIG. 1 is an illustration of powder X-ray diffraction (“PXRD”) pattern of Ponatinib Hydrochloride Form P1 prepared according to example 1.

FIG. 2 is an illustration of powder X-ray diffraction (“PXRD”) pattern of Ponatinib Hydrochloride Form P2 prepared according to example 2.

FIG. 3 is an illustration of powder X-ray diffraction (“PXRD”) pattern of Ponatinib Hydrochloride Form P3 prepared according to example 3.

### DETAILED DESCRIPTION OF THE INVENTION

The present invention provides a crystalline Ponatinib Hydrochloride Form P1 processes for the preparation thereof, and pharmaceutical compositions and formulations comprising the Ponatinib Hydrochloride Form P1, and processes for the preparation of the pharmaceutical compositions and formulations.

In the first embodiment, the present application provides a crystalline Ponatinib Hydrochloride Form P1.

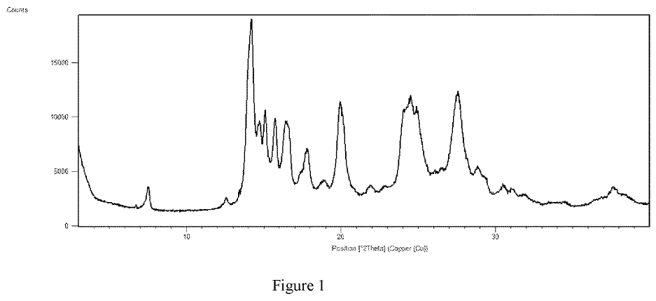

In the second embodiment, the present application provides a crystalline Ponatinib Hydrochloride Form P1 that can be characterized by its PXRD pattern, as illustrated in FIG. 1.

In the third embodiment, the present application provides a crystalline Ponatinib Hydrochloride Form P1, characterized by a PXRD pattern comprising the peaks at about 7.48, 27.54±0.2° 2θ. Further comprising, additional peaks at about 14.22, 15.10 and 15.71±0.2° 2θ.

In the fourth embodiment, the present application provides a process for the preparation of crystalline Ponatinib Hydrochloride Form P1, comprising:
a) providing a mixture of Ponatinib free base or its salt in a suitable solvent comprising acetone;
b) adding hydrochloric acid or a source of chloride anion to the mixture of step a);
c) isolating and recovering the crystalline Ponatinib Hydrochloride Form P1 from the mixture of step b); and
d) drying the said crystalline form.

The mixture comprising Ponatinib free base or its salt in step a) may be a suspension or a solution. The mixture of step a) may be obtained, for example, by providing free base or a salt of Ponatinib of any form in a solvent. The said base or salt may be obtained by a previous step of the process which can be a final reaction and/or purification. If it is intended to obtain a clear solution of Ponatinib free base or its salt, the reaction mixture can be heated to dissolution temperature that can be any temperature as long as the stability of the Ponatinib free base or its salt is not compromised and a substantially clear solution is obtained. For example, the dissolution temperature may range from about 20° C. to about the reflux temperature of the solvent.

If Ponatinib salt is employed in step a) as an input material then it will be different salt from the finally obtained crystalline Ponatinib Hydrochloride Form P1 of step c) and step d).

Solvents employed include, but are not limited to: alcohols, such as, for example, methanol, ethanol, or 2-propanol; esters, such as, for example, ethyl acetate, isopropyl acetate, or t-butyl acetate; ketones such as acetone or methyl isobutyl ketone; ethers, such as, for example, diisopropyl ether, methyl tert-butyl ether, diethyl ether, 1,4-dioxane, THF, or methyl THF; halogenated hydrocarbons, such as, for example, dichloromethane, dichloroethane, chloroform, or the like; hydrocarbons, such as, for example, toluene, xylene, or cyclohexane; nitriles such as acetonitrile; dipolar aprotic solvents such as dimethyl formamide, dimethyl acetamide, dimethyl sulfoxide or like; water; or any mixtures thereof.

Appropriate solvents or non-solvents may be determined by solubility tests in various solvents.

In a preferred embodiment acetone is employed.

Step b) involves addition of hydrochloric acid or source of chloride anion to the mixture of step a).

Suitable solvents are same as that employed in step a) & are inert to the reaction conditions. Non-dissolved particles from a mixture of step b) can be removed suitably by filtration, centrifugation, decantation, or other techniques, such as passing the solution through paper, glass fiber, a particulate bed, or a membrane material.

The reaction can be efficiently completed at room temperature or ambient temperature or if required reaction mass can be heated to elevated temperatures or up to about the reflux temperatures, and maintained for a time from about 10 minutes to about 5 hours or longer. Suitable temperatures for crystallization are from about 0° C. to about 50° C., from about 10 to about 30° C., or any other temperatures may be used. Suitable times for crystallization will vary, and can be from about 10 minutes to about 10 hours, or longer.

Step c) involves isolation and recovery of crystalline Ponatinib Hydrochloride Form P1 from the reaction mixture. The isolation of crystalline Ponatinib Hydrochloride Form P1 may be induced by using conventional techniques known in the art. For example, useful techniques include but are not limited to, concentrating, cooling, stirring, shaking, combining with an anti-solvent, adding seed crystals, evaporation, flash evaporation, simple evaporation, rotational drying, spray drying, thin-film drying including ATFD, freeze-drying, or the like. The solid that is obtained may carry a small proportion of occluded mother liquor containing a higher percentage of impurities and, if desired, the solid may be washed with a suitable solvent to wash out the mother liquor. Evaporation as used herein refers to distilling of solvent almost completely at atmospheric pressure or under reduced pressure. Flash evaporation as used herein refers to distilling of solvent by using a technique includes but is not limited to tray drying, spray drying, fluidized bed drying, thin film drying under reduced pressure, or thin film drying at atmospheric pressure. The recovery of Ponatinib Hydrochloride Form P1 can be done by decantation, centrifugation, gravity filtration, suction filtration and like.

The present invention provides a crystalline Ponatinib Hydrochloride Form P3 processes for the preparation thereof, and pharmaceutical compositions and formulations comprising the Ponatinib Hydrochloride Form P3, and processes for the preparation of the pharmaceutical compositions and formulations.

In the first embodiment, the present application provides a crystalline Ponatinib Hydrochloride Form P3.

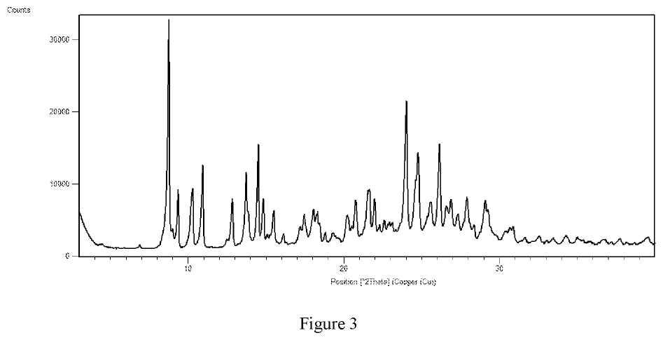

In the second embodiment, the present application provides a crystalline Ponatinib Hydrochloride Form P3 that can be characterized by its PXRD pattern, as illustrated in FIG. 3.

In the third embodiment, the present application provides a crystalline Ponatinib Hydrochloride Form P3, characterized by a PXRD pattern comprising the peaks at about 8.7, 10.9 and 12.8±0.2° 2θ. Further comprising, additional peaks at about 10.23, 14.5, 24 and 26.1±0.2° 2θ.

In the fourth embodiment, the present application provides a process for the preparation of crystalline Ponatinib Hydrochloride Form P3, comprising:
a) providing a mixture of Ponatinib free base or its salt in a suitable solvent comprising acetonitrile;
b) adding hydrochloric acid or a source of chloride anion to the mixture of step a);
c) isolating and recovering the crystalline Ponatinib Hydrochloride Form P3 from the mixture of step b); and
d) drying the said crystalline form.

The mixture comprising Ponatinib free base or its salt in step a) may be a suspension or a solution. The mixture of step a) may be obtained, for example, by providing free base or a salt of Ponatinib of any form in a solvent. The said base or salt may be obtained by a previous step of the process which can be a final reaction and/or purification. If it is intended to obtain a clear solution of Ponatinib free base or its salt, the reaction mixture can be heated to dissolution temperature that can be any temperature as long as the stability of the Ponatinib free base or its salt is not compromised and a substantially clear solution is obtained. For example, the dissolution temperature may range from about 20° C. to about the reflux temperature of the solvent.

If Ponatinib salt is employed in step a) as an input material then it will be different salt from the finally obtained crystalline Ponatinib Hydrochloride Form P3 of step c) and step d).

Solvents employed include, but are not limited to: alcohols, such as, for example, methanol, ethanol, or 2-propanol; esters, such as, for example, ethyl acetate, isopropyl acetate, or t-butyl acetate; ketones such as acetone or methyl isobutyl ketone; ethers, such as, for example, diisopropyl ether, methyl tert-butyl ether, diethyl ether, 1,4-dioxane, THF, or methyl THF; halogenated hydrocarbons, such as, for example, dichloromethane, dichloroethane, chloroform, or the like; hydrocarbons, such as, for example, toluene, xylene, or cyclohexane; nitriles such as acetonitrile; dipolar aprotic solvents such as dimethyl formamide, dimethyl acetamide, dimethyl sulfoxide or like; water; or any mixtures thereof.

Appropriate solvents or non-solvents may be determined by solubility tests in various solvents.

In a preferred embodiment, acetonitrile is employed.

Step b) involves addition of hydrochloric acid or source of chloride anion to the mixture of step a).

Suitable solvents are same as that employed in step a) & are inert to the reaction conditions. Non-dissolved particles from a mixture of step b) can be removed suitably by filtration, centrifugation, decantation, or other techniques, such as passing the solution through paper, glass fiber, a particulate bed, or a membrane material.

The reaction can be efficiently completed at room temperature or ambient temperature or if required reaction mass can be heated to elevated temperatures or up to about the reflux temperatures, and maintained for a time from about 10 minutes to about 5 hours or longer. Suitable temperatures for crystallization are from about 0° C. to about 50° C., from about 10 to about 30° C., or any other temperatures may be used. Suitable times for crystallization will vary, and can be from about 10 minutes to about 10 hours, or longer.

Step c) involves isolation and recovery of crystalline Ponatinib Hydrochloride Form P3 from the reaction mixture. The isolation of crystalline Ponatinib Hydrochloride Form P3 may be induced by using conventional techniques known in the art. For example, useful techniques include but are not limited to, concentrating, cooling, stirring, shaking, combining with an anti-solvent, adding seed crystals, evaporation, flash evaporation, simple evaporation, rotational drying, spray drying, thin-film drying including ATFD, freeze-drying, or the like. The solid that is obtained may carry a small proportion of occluded mother liquor containing a higher percentage of impurities and, if desired, the solid may be washed with a suitable solvent to wash out the mother liquor. Evaporation as used herein refers to distilling of solvent almost completely at atmospheric pressure or under reduced pressure. Flash evaporation as used herein refers to distilling of solvent by using a technique includes but is not limited to tray drying, spray drying, fluidized bed drying, thin film drying under reduced pressure, or thin film drying at atmospheric pressure. The recovery of Ponatinib Hydrochloride Form P3 can be done by decantation, centrifugation, gravity filtration, suction filtration and like.

The crystalline Ponatinib Hydrochloride Form P1 or Form P3 of the present invention if desired can be purified by re-crystallization from an appropriate re-crystallization solvent or mixture of solvents by methods customary to one of skill in the art. If required, the process further comprises, at a suitable stage, removing or separating any undesired material or impurities, and finally, optionally, the salts may be washed and/or dried.

The resulting solid may be further dried. Drying may be suitably carried out by using equipment such as a tray dryer, vacuum oven, air oven, fluidized bed dryer, spin flash dryer, flash dryer, or the like, at atmospheric pressure or under reduced pressure. Drying may be carried out at temperatures less than about 100° C., less than about 60° C., less than about 40° C., or any other suitable temperatures, at atmospheric pressure or under reduced pressure, and in the presence or absence of an inert atmosphere such as nitrogen, argon, neon, or helium. The drying may be carried out for any desired time periods to achieve a desired purity of the product, such as, for example, from about 1 hour to about 15 hours, or longer.

Once obtained, crystals of crystalline Ponatinib Hydrochloride Form P1 or Form P3 may be used as the nucleating agent or “seed” crystals for subsequent crystallizations of salts of crystalline Ponatinib Hydrochloride Form P1 or Form P3 from solutions.

The crystalline Ponatinib Hydrochloride Form P1 and Form P3 of the present application may be characterized by means of Powder X-ray Diffraction Pattern (PXRD). Other techniques, such as solid state NMR, Fourier Transform Infrared (FTIR), differential scanning calorimetry (DSC) may also be used.

Ponatinib employed as a starting material for preparation of crystalline Ponatinib Hydrochloride Form P1 and Form P3 can be obtained by any processes known in the art, including processes disclosed in U.S. Pat. Nos. 8,114,874, 9,493,470 which are incorporated herein by reference in their entireties, as well as by other processes known in the art.

The compound of this application is best characterized by the X-ray powder diffraction pattern determined in accordance with procedures that are known in the art. PXRD data reported herein was obtained using CuKα radiation, having the wavelength 1.5406 Å and were obtained using a PANalytical X'Pert PRO instruments. For a discussion of these techniques see J. Haleblain, J. Pharm. Sci. 1975 64:1269-1288, and J. Haleblain and W. McCrone, J. Pharm. Sci. 1969 58:911-929.

Generally, a diffraction angle (20) in powder X-ray diffractometry may have an error in the range of ±0.2°. Therefore, the aforementioned diffraction angle values should be understood as including values in the range of about ±0.2°. Accordingly, the present application includes not only crystals whose peak diffraction angles in powder X-ray diffractometry completely coincide with each other, but also crystals whose peak diffraction angles coincide with each other with an error of about ±0.2°. Therefore, in the present specification, the phrase “having a diffraction peak at a diffraction angle (2θ±0.2°) of 7.9°” means “having a diffraction peak at a diffraction angle (2θ) of 7.7° to 8.1°”. Although the intensities of peaks in the x-ray powder diffraction patterns of different batches of a compound may vary slightly, the peaks and the peak locations are characteristic for a specific polymorphic form. Alternatively, the term “about” means within an acceptable standard error of the mean, when considered by one of ordinary skill in the art. The relative intensities of the PXRD peaks can vary depending on the sample preparation technique, crystal size distribution, various filters used, the sample mounting procedure, and the particular instrument employed. Moreover, instrument variation and other factors can affect the 2-theta values. Therefore, the term “substantially” in the context of PXRD is meant to encompass that peak assignments can vary by plus or minus about 0.2 degree. Moreover, new peaks may be observed or existing peaks may disappear, depending on the type of the machine or the settings (for example, whether a Ni filter is used or not).

Optionally crystalline Ponatinib Hydrochloride Form P1 or Form P3 of the present invention can be subjected to particle size reduction by conventional techniques like jet-milling, micronization and like to obtain suitable particle size distribution. The D10, D50, and D90 values are useful ways for indicating a particle size distribution. D90 refers to at least 90 volume percent of the particles having a size smaller than the said value. Likewise, D10 refers to 10 volume percent of the particles having a size smaller than the said value. D50 refers to 50 volume percent of the particles having a size smaller than the said value. Methods for determining D10, D50, and D90 include laser diffraction, such as using equipment from Malvern Instruments Ltd. of Malvern, Worcestershire, United Kingdom.

In the fifth embodiment, the present application provides a pharmaceutical composition comprising crystalline Ponatinib Hydrochloride Form P1 or Form P3 and one or more pharmaceutically acceptable excipients.

Such further excipients and adjuvants are known to the person skilled in the art and may include one or more fillers; diluents, for example microcrystalline cellulose, lactose, mannitol, dibasic calcium phosphate, pregelatinized starch and the like; binders such as PVP, HPMC, HPC and the like; disintegrants, for example, sodium starch glycolate, crospovidone, croscarmellose sodium and the like; lubricants, for example, magnesium stearate, sodium stearyl fumarate and the like; sweeteners, for example, sucrose, saccharin and the like; flavoring agents, for example, peppermint, methyl salicylate, orange flavoring and the like; colorants; preservatives; buffers; and/or other excipients depending on the dosage form used.

The pharmaceutical compositions of the present invention are generally administered orally to patients, which include, but are not limited to, mammals, for example, humans, in the form of, for example, a hard or soft gelatin capsule, a tablet, a caplet, pills, granules or a suspension. The pharmaceutical dosage form can be prepared by methods known in the art, such as direct compression or wet granulation or direct compression. The compression of the blend to tablet cores can be carried out using a conventional tabletting machine or a rotary compression machine. The tablet cores may vary in shape and can be, for example, round, oval, oblong, cylindrical or any other suitable shape. The cores may also vary in size depending on the concentration of the therapeutic agent.

The pharmaceutical dosage form according to the present invention may be is coated with one or more coating materials or uncoated. The coating materials are not particularly limited and are known to the person skilled in the art.

Certain specific aspects and embodiments of the present application will be explained in greater detail with reference to the following examples, which are provided by way of illustration only and should not be construed as limiting the scope of the application in any manner.

### Definitions

The following definitions are used in connection with the present application unless the context indicates otherwise. Polymorphs are different solids sharing the same molecular formula, yet having distinct physical properties when compared to other polymorphs of the same formula. The abbreviation “MC” mean moisture content. Moisture content can be conveniently measured, for example, by the Karl Fischer method.

“Crystalline form” as used herein refers to a solid state wherein the crystalline content with in the said solid state is at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

A crystal form may be referred to herein as being characterized by graphical data “substantially as depicted” in a Figure. Such data include, for example, powder X-ray diffractograms, infrared spectra and DSC thermograms. The graphical data potentially provides additional technical information to further define the respective solid state form which can not necessarily or easily be described by reference to numerical values for peak positions and/or relative intensities. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown solid state form and confirm whether the two sets of graphical data are characterizing the same solid state form or two different solid state forms.

Where this document refers to a material, such as in this instance, crystalline Ponatinib Hydrochloride Form P1 or Form P3, by reference to patterns, spectra or other graphical data, it may do so by qualifying that they are “substantially” shown or as depicted in a Figure, or by one or more data points. By “substantially” used in such a context, it will be appreciated that patterns, spectra and other graphical data can be shifted in their positions, relative intensities and/or values due to a number of factors known to those of skill in the art.

In addition, where a reference is made to a figure, it is permissible to, and this document includes and contemplates, the selection of any number of data points illustrated in the figure which uniquely define that solid state form, within any associated and recited margin of error, for purposes of identification.

As used herein, the term “isolated” corresponds to product or solid state form thereof that is physically separated from the reaction mixture in which it is formed.

All percentages and ratios used herein are by weight of the total composition, unless the context indicates otherwise. All temperatures are in degrees Celsius unless specified otherwise and all measurements are made at 25° C. and normal pressure unless otherwise designated. The present disclosure can comprise the components discussed in the present disclosure as well as other ingredients or elements described herein.

As used herein, “comprising” means the elements recited, or their equivalents in structure or function, plus any other element or elements which are not recited. The terms “having” and “including” are also to be construed as open ended unless the context suggests otherwise.

All ranges recited herein include the endpoints, including those that recite a range “between” two values.

Terms such as “about,” “generally,” “substantially,” or the like are to be construed as modifying a term or value such that it is not an absolute. Such terms will be defined by the circumstances and the terms that they modify, as those terms are understood by those of skill in the art. This includes, at very least, the degree of expected experimental error, technique error and instrument error for a given technique used to measure a value.

When a molecule or other material is identified herein as “pure”, it generally means, unless specified otherwise, that the material is 99% pure or more, as determined by methods conventional in art such as high performance liquid chromatography (HPLC) or optical methods. In general, this refers to purity with regard to unwanted residual solvents, reaction byproducts, impurities, and unreacted starting materials. “Substantially” pure means, the same as “pure except that the lower limit is about 98% pure or more and likewise, “essentially” pure means the same as “pure” except that the lower limit is about 95% pure.

As used herein, the term “room temperature” refers to a temperature of from about 20° C. to about 35° C., from about 25° C. to about 35° C., from about 25° C. to about 30° C., or for example, about 25° C.

As used herein, the term “overnight” refers to a time interval from about 14 hours to about 24 hours, or about 14 hours to about 20 hours, for example, about 16 hours.

The “polymer” or “carrier” or “excipient” as used herein interchangeably refers to any substance or mixture of substances which are pharmaceutically acceptable inactive ingredients.

Certain specific aspects and embodiments of the present application will be explained in greater detail with reference to the following examples, which are provided only for purposes of illustration and should not be construed as limiting the scope of the application in any manner.

### EXAMPLES

### Example 1: Preparation of Crystalline Ponatinib Hydrochloride Form P1

A flask was charged with Ponatinib free base (1 g), acetone (30 mL) at room temperature for dissolution followed by cooling to ˜15° C. To this mixture, solution of 10% aq. hydrochloric acid (0.6 microliter) in acetone (10 mL) was slowly added at the same temperature. The resulting mixture was stirred at room temperature for about 3 hours. The obtained solid was filtered followed by vacuum drying at 50° C. for about 6-7 hours to afford the title compound as depicted in FIG. 1.

### Example 2: Preparation of Crystalline Ponatinib Hydrochloride Form P2

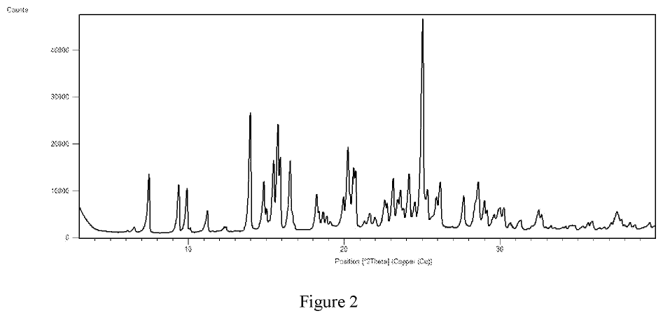

Ponatinib HCl (500 mg) and propionic acid (0.5 mL) was grinded in a mortar-pestle followed by addition of MTBE (2 mL) and further grinding. The obtained solid was subjected to drying under VTD at about 60° C. to afford the title compound as depicted in FIG. 2.

### Example 3: Preparation of Crystalline Ponatinib Hydrochloride Form P3

A flask was charged with Ponatinib free base (15 g), acetonitrile (150 mL) at room temperature for dissolution followed by cooling to about 15° C. To this mixture, solution of 10% aq. hydrochloric acid (9.6 mL) was slowly added at the same temperature. The resulting mixture was stirred at room temperature for about 22 hours. The obtained solid was filtered followed by slurring in n-heptane followed by vacuum drying at 50° C. for about 2 hours to afford the title compound, PXRD as depicted in FIG. 3.

1. A crystalline Ponatinib Hydrochloride Form P1.

2. A process for the preparation of crystalline Ponatinib Hydrochloride Form P1 of claim 1, comprising:
a) providing a mixture of Ponatinib free base or its salt in a suitable solvent comprising acetone;
b) adding hydrochloric acid or a source of chloride anion to the mixture of step a);
c) isolating and recovering the crystalline Ponatinib Hydrochloride Form P1 from the mixture of step b); and
d) drying the said crystalline form.

3. The process of claim 2, where the suitable solvent is acetone.

4. The process of claim 2, where crystalline Ponatinib Hydrochloride Form P1 is characterized by its PXRD pattern, as illustrated in FIG. 1.

5. The process of claim 2, where crystalline Ponatinib Hydrochloride Form P1 is characterized by a PXRD pattern comprising the peaks at about 7.48, 27.54±0.2° 20.

6. A crystalline Ponatinib Hydrochloride Form P3.

7. A process for the preparation of crystalline Ponatinib Hydrochloride Form P3 of claim 6, comprising:
a) providing a mixture of Ponatinib free base or its salt in a suitable solvent comprising acetonitrile;
b) adding hydrochloric acid or a source of chloride anion to the mixture of step a);
c) isolating and recovering the crystalline Ponatinib Hydrochloride Form P3 from the mixture of step b); and
d) drying the said crystalline form.

8. The process of claim 7, where the crystalline Form P3 is further slurried with n-heptane.

9. The process of claim 7, where crystalline Ponatinib Hydrochloride Form P3 is characterized by its PXRD pattern, as illustrated in FIG. 3.

10. The process of claim 7, where crystalline Ponatinib Hydrochloride Form P3, is characterized by a PXRD pattern comprising the peaks at about 8.7, 10.9 and 12.8±0.2° 2θ.

11. A pharmaceutical composition comprising crystalline Ponatinib hydrochloride Form P1 of claim 1 and pharmaceutically acceptable excipients.

12. A pharmaceutical composition comprising crystalline Ponatinib hydrochloride Form P3 of claim 6 and pharmaceutically acceptable excipients.
